(19) United States
(12) Patent Application Publication
(10) Pub. No.: US 2003/0023234 A1
(43) Pub. Date: Jan. 30, 2003

(54) Laser light irradiation apparatus

(76) Inventor: Norio Daikuzono, Napa, CA (US)

Correspondence Address: MCDERMOTT WILL & EMERY, 600 13TH STREET, N.W., WASHINGTON, DC, 20005-3096, US

(21) Appl. No.: 09/916,202

(22) Filed: Jul. 27, 2001

(51) Int. Cl. A61B 18/22

(52) U.S. Cl. 606/9; 606/16; 606/17

(57) ABSTRACT

The present invention relates to a laser light irradiating apparatus in which the laser light is not liable to act upon the tissue other than the target tissue to be irradiated with Laser light and gives less heat feeling to the living tissue to be irradiated. The laser light irradiating apparatus comprises an optical fiber 3 having its rear end upon which the laser light is incident from said laser light supplying means and its front end from which the laser light is emitted; a handpiece 1 for holding said optical fiber 3; and means for injecting a laser light absorbing liquid in which a laser light absorbing material which causes photodecomposition thereof with said laser light is dispersed toward an target position to be irradiated on which the laser light L emitted from the front end of said optical fiber 3 is impinged.

### TECHNICAL FIELD TO WHICH THE INVENTION PERTAINS

The present invention relates to a laser light irradiation apparatus for irradiating a living tissue with laser light and in particular to a laser light irradiation apparatus for irradiating the living tissue with laser light such as the pulsed layer light such as Nd:YAG in combination with a laser light absorbing liquid containing a material which absorbs the laser light to cause photodecomposition for incision and evaporation of the living tissue.

### BACKGROUND OF THE INVENTION

In field of surgery, dentistry as well as cosmetic surgery, irradiation of the living tissue with laser light f or incision and evaporation of the living tissue has been conducted. For example, U.S. Pat. No. 4,818,230 discloses that the caries of a tooth is irradiated with pulsed laser light for the treatment thereof. The pulsed laser light has a power of 0. 1 to 100 mJ (milli Joules) per one pulse. Yttrium-Aluminium-Garnet laser light is used as this laser. The laser light has less energy.

However, prior art laser light irradiating apparatus has serious problems that the peripheral tissue which is not a target to be treated (including a deep portion) will be carbonized and solidified and that a patient to be irradiated feels pain due to the fact that (1) the laser light acts on the inner non-target tissue in deep area as well as the tissue to be irradiated and that (2) heat which is generated in the irradiated target tissue will conduct to the peripheral non target tissue.

Blowing an air toward the tissue to be irradiated with laser light for cooling it in order to solve the problems is known. Impinging of the laser light upon the peripheral non target tissue is inevitable and the cooling effect is practically less.

Influence of the laser light upon the peripheral non-target tissue and heat feeling which the patient to be irradiated can endure should be considered. Since the laser light having an excessive power cannot be impinged, small number of applicable treatment methods is an issue.

Therefore, it is a main object of the present invention to provide a laser light irradiating apparatus in which the laser light is not liable to act upon the tissue other than the target tissue to be irradiated and gives less heat feeling to the patient to be treated.

### SUMMARY OF THE INVENTION

The present invention provides a laser light irradiating apparatus for irradiating the living tissue with laser light from laser light supplying means, characterized in that said apparatus comprises an optical fiber having its rear end upon which the laser light is incident from said laser light supplying means and its front end from which the laser light is emitted; a handpiece for holding said optical fiber; and means for injecting a light absorbing liquid in which a laser light absorbing material which causes photodecomposition thereof with said laser light is dispersed toward an target position to be irradiated on which the laser light emitted from the front end of said optical fiber is impinged.

By thus forming the laser light irradiating apparatus, the target tissue can be irradiated with laser light while the laser light absorbing liquid is injected thereon. The laser light which is incident upon the target tissue will be absorbed by the laser light absorbing material existing in the target tissue to be irradiated. As a result, the laser light absorbing material will be photodecomposed, and in association with this, the living tissue around the laser light absorbing material will be simultaneously decomposed, so that breaking, melting and evaporation of the living tissue in interest occurs.

The laser light which is incident upon the surface area of the tissue which intersects with the effective irradiation area is almost absorbed or scattered by the laser light absorbing material existing in the area in interest, so that it passes through the surface area in interest and will not act on the non-target tissue in the inside thereof. Therefore, the non-target tissue in the inner side will be scarcely influenced by the laser light. At the surface area of the tissue which intersects with the effective irradiation area, heat is generated due to the photodecomposition of the laser light absorbing material. The heat will be removed by the cooling effect of the dispersion liquid of the laser light absorbing liquid. Since the laser light absorbing liquid which is supplied to the periphery of the surface area which intersects with the effective irradiation area Z is not effectively irradiated with the laser light, it functions as only cooling liquid. Accordingly, the heat feeling which is given to the living body to be irradiated is very small

The laser light absorbing liquid including laser light absorbing particles having a particle size of 10 microns or more which is formed from one or more materials selected from the group of titanium oxide, manganese dioxide, iron oxide and carbon are preferably dispersed in a dispersion matrix liquid including water as a main constituent and carboxymethyl cellulose which is added so that the concentration of the carboxymethyl cellulose is 0.1% or more. When the target tissue is the ivory of a tooth, particularly preferable laser absorbing particles are titanium oxide particles. If carbon particles were used, the target tissue and the peripheral tissue would be colored into black. Since the titanium oxide particles are white in color, the treated tissue is finished beautiful. The dispersion matrix liquid in which carboxymethylcellulose is added to water in such an amount that the concentration of carboxymethylcellulose is 0.1% or more has such a moderate viscosity that the laser light absorbing particles will not be sedimented. Such a laser light absorbing liquid can keep the excellent dispersion condition of the laser light absorbing particles, so that the laser light absorbing particles are uniformly supplied to the target tissue when the laser light absorbing liquid is injected to the target tissue.

It is preferable that the laser light used in the present invention has a wave length of 0.7 to 1.7 μm in, for example, dental application. The preferred laser light is Nd:YAG laser light. In the present invention, KTP laser, Alexandra light laser may be used. It is preferable that the pulsed laser light has a pulse width of 100 ms or less and an energy of 300 mJ or more per one pulse and a repetition rate of 50 pps or less per one second.

The specific structure of the apparatus is proposed in which said apparatus has a guide tube at the front end of a handpiece, an optical fiber extends through the guide tube and projects beyond the front end of the guide tube and the laser light absorbing liquid flows through a space between the guide tube and the optical fiber and is injected from an opening at the front end of the guide tube.

By thus forming the apparatus, almost all laser light absorbing liquid which is injected from the absorbing liquid injecting means reaches at the target tissue to be irradiated with laser light while surrounding the periphery of the laser light emitted from the front end of the optical fiber and then diverses along the target tissue to be irradiated. Accordingly, the effective irradiation area of the laser light occurs only in the front of the front end of the optical fiber and the peripheral area around the laser light is cooled. Observing in the atmosphere, little laser light absorbing liquid flows at the effective irradiation area. Thus, the laser light is scarcely absorbed by the laser light absorbing liquid at the effective irradiation area. This implies that it is possible to locally irradiate the target tissue with the laser light having a high energy while the peripheral tissue is cooled (elevation in temperature is prevented).

### BRIEF DESCRIPTION OF THE DRAWINGS

FIG. 1 is a schematic explanatory view of the laser light irradiating apparatus of the present invention;

FIG. 2 is an enlarged schematic view of the front end of the apparatus in FIG. 1;

FIG. 3 is an enlarged view of FIG. 2;

FIG. 4 is a schematic explanatory view of the other laser light irradiating apparatus of the present invention;

FIG. 5 is an enlarged schematic view of the front end of the apparatus in FIG. 4;

FIG. 6 is an enlarged view of FIG. 5;

FIG. 7 is a partly cut away front view showing another example of the front end of the apparatus;

FIG. 8 is an explanatory view showing the manner in which laser light irradiation is conducted;

FIG. 9 is an explanatory view showing another manner in which laser light irradiation is conducted;

FIG. 10 is an explanatory view showing an exemplary treatment of a tooth; and

FIG. 11 is an enlarged view showing the manner in which the laser light irradiation is conducted by a prior art apparatus.

### PREFERRED MODE OF EMBODYING THE PRESENT INVENTION

Now, the preferable modes of embodying the present invention will be described in more detail with reference to the drawings.

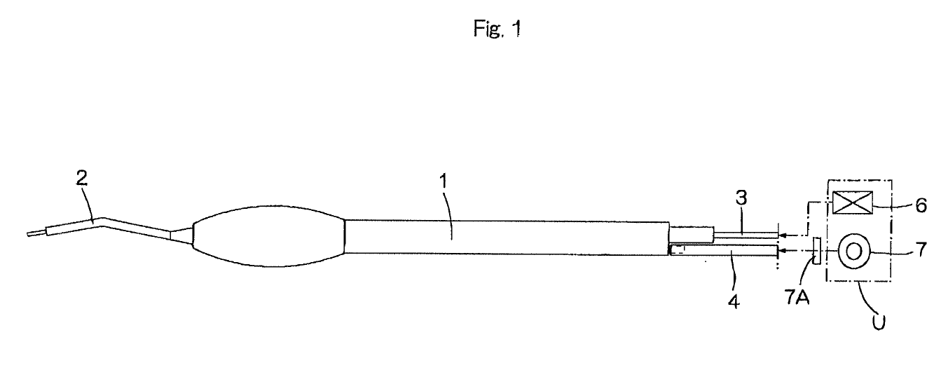

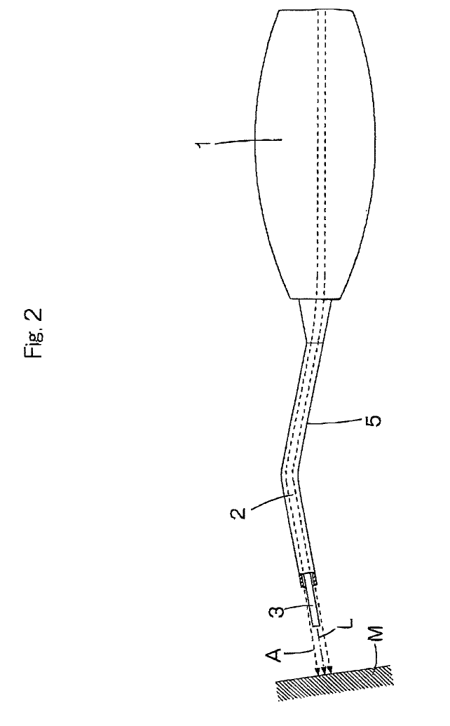

Firstly, the fundamental concept of the present invention will be described with reference to FIGS. 1 and 2. The laser light transmitting member of the present invention includes an optical fiber 3. Laser light from laser light supplying means is incident upon the rear end of the optical fiber 3 and passage through the inside thereof and is emitted from the front end of the optical fiber 3 so that the laser light is incident upon a target tissue such as tooth.

The optical fiber 3 is held by a handpiece 1. The handpiece is hollow therein and has a guide tube 2 at its front end. The guide tube 2 is communicated with the inside of the handpiece 1. The optical fiber 3 is inserted into the guide tube 2 at the rear end portion of the handpiece 1 and extends through the handpiece 1 and the guide tube 2 and is exposed from the front end of the guide tube 2.

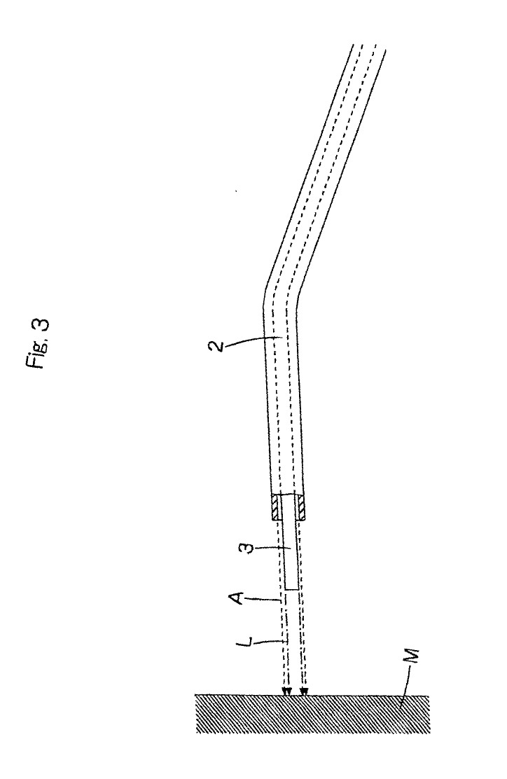

As clearly shown in FIG. 3, a space is formed between the guide tube 2 and the optical fiber 3. A laser light absorbing liquid A which is pumped from the rear end of the handpiece 1 and through a feed tube 4 (refer to FIG. 1) which is made of a flexible plastic passes through the handpiece 1 and is injected upon a target tissue M in a coaxial manner from a space between the guide tube 2 and the optical fiber 3 in a forward direction. The laser light absorbing liquid including laser light absorbing particles having a particle size of 10 microns or more which is formed from one or more materials selected from the group of titanium oxide, manganese dioxide, iron oxide and carbon are preferably dispersed in a dispersion matrix liquid including water as a main constituent and carboxymethylcellulose which is added so that the concentration of the carboxymethylcellulose is 0.1% or more is used. Although the carboxymethyl cellulose is added for the purpose of adjusting the viscosity, it may be omitted or may be replaced with the other viscosity adjusting agent. Emulsifying agent or preservatives may be added to the dispersing liquid for enhancing the long term stability and preservation ability.

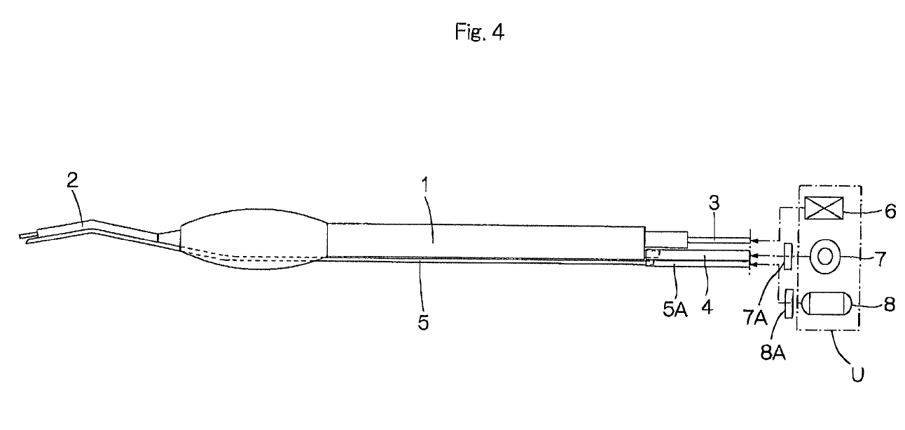

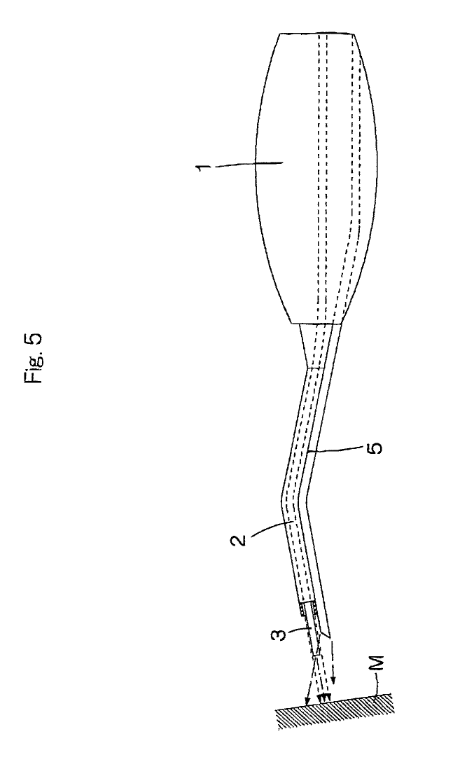

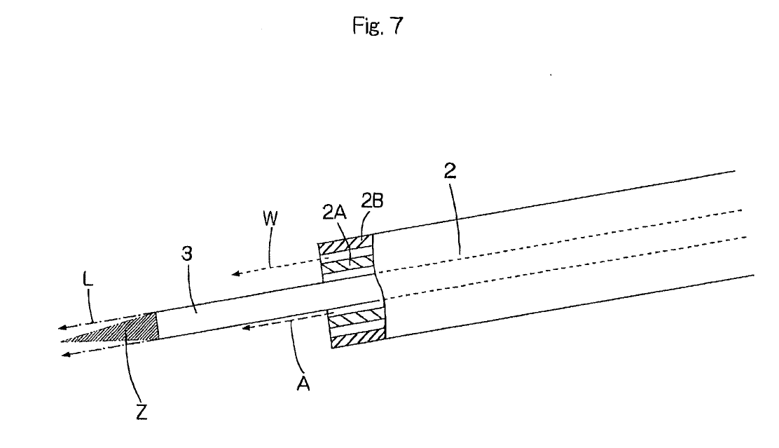

In order to enhance the cooling effect, means for injecting a cooling medium comprising gas such as air, liquid such as water or the mixture thereof, and containing now laser light absorbing material (hereinafter only referred to as cooling medium) upon the target tissue may be provided according to needs. This example is shown in FIGS. 4 to 6. A cooling medium tube 5 of metal having a small diameter is provided below and along the handpiece 1 and the guide tube 2 so that it is made integral with the handpiece 1 and the guide tube 2 by means of adhesive or welding. The cooling medium tube 5 is linked at its rear end with a cooling medium tube 5A of a flexible plastic. Since the opening at the front end of the cooling medium tube 5 is located closer to the base portion of the optical fiber 3 with respect to the front end thereof as shown in the drawings, it is capable of supplying the cooling medium W in a substantially coaxial manner in a forward direction from the periphery of the front end of the optical fiber 3. Alternatively, a structure may be adopted in which a double coaxial guide tube 2 having inner and outer tubes 2A and 2B is provided, and the optical fiber 3 is inserted into the inner tube 2A so that a space between the inner tube 2A and the optical fiber 3 is used as a passage for supplying the laser light absorbing liquid A and a space between the inner and outer tubes 2A and 2B is used as a passage for supplying the cooling medium W as shown in FIG. 7. Although not shown, the number of the cooling medium tube may be increased or double or more coaxial tubes may be used if the cooling liquid and cooling gas are separately supplied.

In the example shown in FIG. 1, the optical fiber 3 and liquid supply tube 4 can be removably linked with the connecting openings of the laser light irradiating unit U (the details thereof not shown). The light irradiating unit U comprises the laser light supply means such as Nd:YAG laser light generator 6 and its controller (not shown) for the output and the liquid supply pump 7.

The laser light irradiating unit U has a control panel. The parameters such as one pulse width, the energy per one pulse (in unit of mJ), pulse repetition rate of the pulse laser light and the flow rate of the supplied air A per one minute and the flow rate of supplied water per one minute can be selectively preset by operating the display unit of the control panel. A foot switch 7A for supplying the laser light absorbing liquid is connected to the laser light irradiating unit U. The selection between injecting and stopping modes of the laser light absorbing liquid may be switched by turning on or off the foot switch 7A for supplying liquid. Selection between on or off of the laser light irradiation may be switched by the switch or the foot switch (not shown) which is provided on the handpiece 1. Although not shown, the switch for supplying laser light absorbing liquid and the switch for laser light irradiation may be made common.

When the means for spaying the cooling medium upon the target tissue is provided as shown in FIG. 4, the laser light irradiating unit U is provided with a cooling medium supply compressor or pump 8 and the cooling medium supply foot switch 8A is connected thereto. Selection between ejection and stopping modes of the laser light absorbing liquid can be switched by turning on/off the cooling medium supply foot switch 8A. The cooling medium supply switch may be made common with either one or both of the laser light absorbing liquid supply switch and the laser irradiation switch.

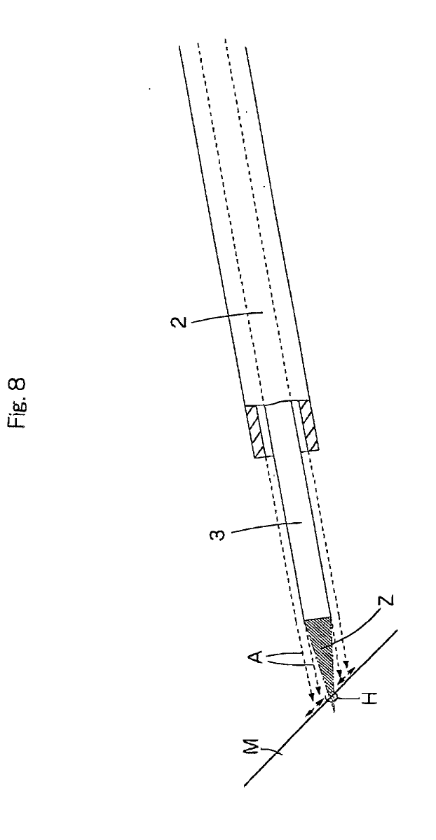

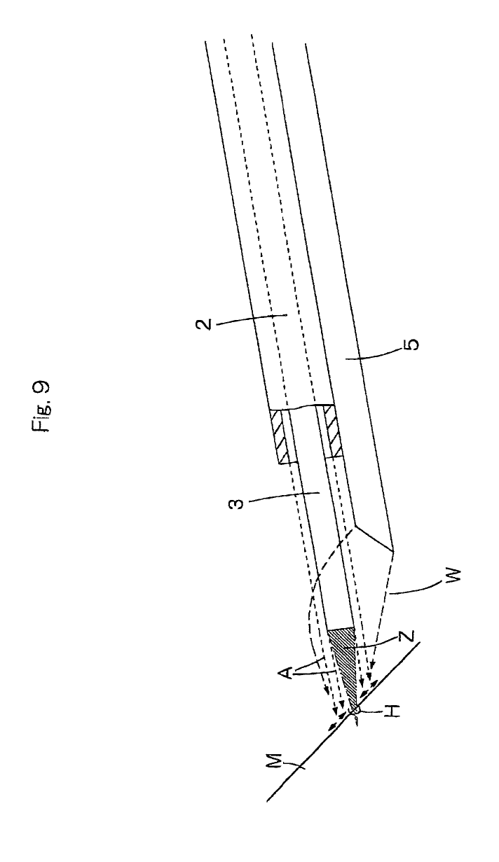

Now, the manner of laser light irradiation will be described. Observing in the atmosphere, the optical fiber 3 is pointed toward the tissue and the irradiation with the pulsed laser light L and supplying of the laser light absorbing liquid A is commenced as shown in FIG. 3. Thereafter, the laser light absorbing liquid A flows along the outer periphery of the optical fiber 3 until it reaches the front end of the optical fiber 3 and then is injected in a forward direction in a cylindrical manner surrounding the effective irradiation area Z of the laser light from the front end of the optical fiber 3 as shown in FIG. 8. If the cooling medium W is supplied simultaneously with this, the cooling medium W flows around the front end of the optical fiber 3, resulting in that it is entrained with the laser light absorbing liquid A and is caused to flow in a forward direction as shown in FIG. 9. Therefore, the cooling medium W also hardly flows into the effective irradiation area Z.

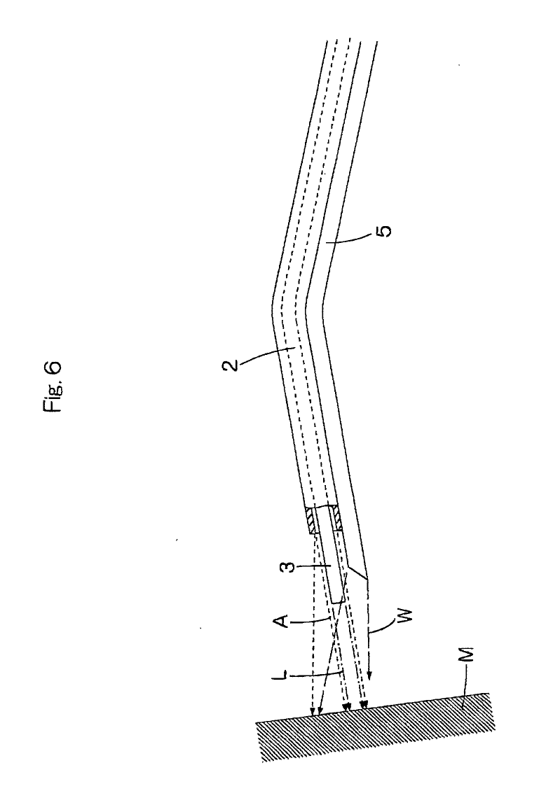

If the incision and evaporation or solidification of the tissue M is achieved by the irradiation with the pulsed laser light L, the tissue M is irradiated with the pulsed laser light L (refer to FIGS. 3 and 6) while the laser light absorbing liquid A is supplied under a condition in which the front end of the optical fiber 3 is close to the tissue in such a manner that the surface of the tissue M is within the laser light effective irradiation area Z as shown in FIGS. 8 and 9. The supplied laser light absorbing liquid A passes along the periphery of the laser light effective irradiation area Z and reaches the surface of the tissue M and is spread uniformly over the surface area and the periphery area of the tissue M which intersect with the effective irradiation area Z over the surface of the tissue M. This causes the laser light absorbing material in the absorbing liquid A is also uniformly spread to the surface area and the periphery area of the tissue. On the other hand, the laser light L passes through the cavity for the laser light absorbing liquid A which is cylindrical and reaches the surface area of the tissue which intersects with the effective irradiation area Z without being attenuated. At the surface area of the tissue M which intersects with the effective irradiation area Z, the laser light absorbing material which exists in the surface area will absorb the laser light. As a result, the laser light absorbing material will be photodecomposed, and in association with this, the living tissue around the laser light absorbing material will be simultaneously decomposed, so that breaking, melting and evaporation of the living tissue in interest occurs.

The laser light L which is incident upon the surface area of the tissue M which intersects with the effective irradiation area Z is almost absorbed or scattered by the laser light absorbing material existing in the area in interest, so that it passes through the surface area in interest and will not act on the non-target tissue in the inside thereof. Therefore, the non-target tissue in the inner side will be scarcely influenced by the laser light. At the surface area of the tissue M which intersects with the effective irradiation area Z, heat is generated due to the photodecomposition of the laser light absorbing material. The heat will be removed by the cooling effect of the dispersion liquid of the laser light absorbing liquid A.

Since the laser light absorbing liquid A which is supplied to the periphery of the surface area which intersects with the effective irradiation area Z is not effectively irradiated with the laser light, it functions as only cooling liquid. Accordingly, the heat feeling which is given to the living body to be irradiated is very small. If the cooling medium W is supplied as shown in FIG. 9, the medium W is supplied to the periphery of the surface area which intersects with the effective irradiation area Z, so that cooling is constantly conducted. As a result, the temperature at the periphery of the surface area of the tissue M will not be elevated.

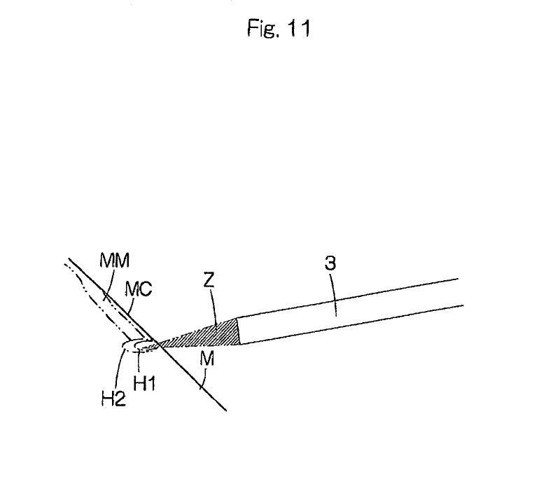

In accordance with the laser light irradiating apparatus of the present invention, a portion H, at which the temperature will be elevated is localized as shown in FIGS. 8 and 9, so that carbonation and solidification of the peripheral tissue will scarcely occur. In contrast to this, in the prior art apparatus in which the laser light absorbing liquid is not injected, the laser light will act in a depth direction as shown in FIG. 11. Accordingly, the high temperature portions H1 and H2 extend in a depth direction and heat will be conducted from the laser light irradiated portion to the periphery thereof, so that the carbonized portion MC and solidified portion MS occur in the periphery of the laser light irradiated area.

The above-mentioned phenomenon has been presumed from a result of measurement of the tissue temperature using a thermal infra-red ray camera on irradiation of the actual tissue with the pulsed laser light L while the distance between the optical fiber 3 and the tissue is changed.

It is preferable that the laser light used in the present invention has a wave length of 0.7 to 1.7 μm in, for example, dental application. The preferred laser light is Nd:YAG laser light. In the present invention, KTP laser, Alexandra light laser may be used. It is preferable that the pulsed laser light has a pulse width of 100 ms or less and an energy of 300 mJ or more per one pulse and a repetition rate of 50 pps or less per one second. Within the range of these conditions, hemostasis, incision and evaporation of the soft tissue, hemostasis, sterility and drainage of the gingival, inflected root canal treatment, removal and cleaning of tartar, cutting and opening of the enamelum, cutting of opening of the ivory, bonding of crowns, and fixing by welding of metal implant screw can be conducted.

Selective removal of the caries portion of the tooth can be advantageously conducted by using Nd:YAG laser light. Enamel portion which constitutes the ivory is mainly formed from inorganic hydroxyapatite and will not absorb the light having a wave length in the vicinity of 1.06 μm. Since the caries portion which is generated in the enamel contains much protein component which is the modified enamel and is colored, the caries portion can be selectively removed on irradiation of it with Nd:YAG laser light having a wave length in the vicinity of 1.06 μm which causes the absorption of protein. This reaction occurs when the caries portion exhibits a high absorption efficiency for the laser light for a short period of time. Whether this reaction occurs or not mainly depends upon the pulse width, the energy of laser light per one pulse and repetition rate of the pulses per one second. The width of one pulse is preferably 10 ms or less. If it is larger than this value, the peak power in one pulse will be lowered, so that absorption of the laser light is difficult. If the laser light energy per one pulse is low, that is 300 mJ or less per one pulse, absorption of the laser light is difficult. If the repetition rate of pulses exceeds 50 pps per one second, the caries portion can be removed but heat will remain, so that the temperature of the whole of ivory will be elevated. Pulpitis may occur.

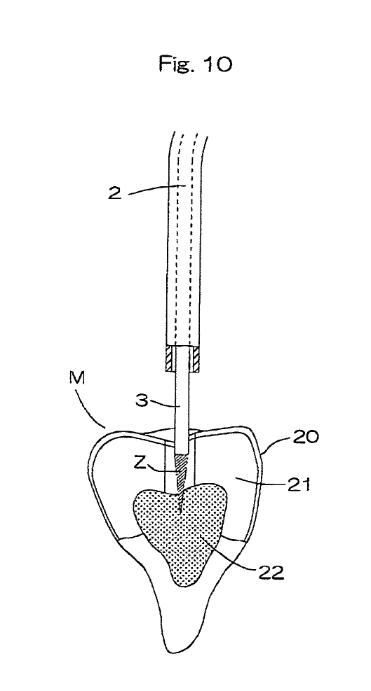

FIG. 10 shows a treatment for removing the caries portion in the dental pulp 22, which has promoted from the enamel 20 of tooth M to the dental pulp 22 through the ivory 21 by cutting the ivory 21. Irradiation of the dental pulp 22 with pulsed laser light will scaresly give pain to the patient. In the present invention, a light guide chip which transmits the laser light may be connected to or disposed on the front end of the optical fiber. Whether the front face of the optical fiber 3 is contact with the surface of the tissue M or separated therefrom depends upon the type of treatment.

Selection between only air-supply, simultaneous supply of air and water, only water supply and neither supply can be switched depending upon the type of treatment. Supply of both air and water, has remarkably high effect of cooling the irradiation portion with the laser light. Supply of air will remove incised or cut tissue or evaporated or solidified material to cause new tissue to be exposed. Supply of only air or supply of only water may be conducted. Since for removal of tartar, the irradiation of the tartar with laser light in the presence of water to cause cavitation in the tartar is effective, supply of only waver is preferable. In contrast to this, simultaneous supply of air and water has a remarkably high cooling effect. Liquid such as alcohol may be used in lieu of water.

In the course of supply of laser light absorbing liquid, supply of cooling medium can not be continuously conducted, but intermittently conducted. In order to conduct such an intermittent supply of cooling medium, supply passage for the cooling medium may be turned on or off by the handpiece or foot switch.

As mentioned above, the laser light is liable to act upon the tissue other than the target tissue to be irradiated with laser light and heat feeling which is given to the living body to be irradiated becomes less in the laser light irradiating apparatus of the present invention.

1. A laser light irradiating apparatus for irradiating the living tissue with laser light from laser light supplying means, characterized in that said apparatus comprises
an optical fiber having its rear end upon which the laser light is incident from said laser light supplying means and its front end from which the laser light is emitted;
a handpiece for holding said optical fiber; and
means for injecting a laser light absorbing liquid in which a laser light absorbing material which causes photodecomposition thereof with said laser light is dispersed toward an target position to be irradiated on which the laser light emitted from the front end of said optical fiber is impinged.

2. A laser light irradiating apparatus as defined in claim 1 wherein said laser light absorbing liquid including laser light absorbing particles having a particle size of 10 microns or more which is formed from one or more materials selected from the group of titanium oxide, manganese dioxide, iron oxide and carbon are dispersed in a dispersion matrix liquid including water as a main constituent and carboxymethyl cellulose which is added so that the concentration of the carboxymethyl cellulose is 0.1% or more is used.

3. A laser light irradiating apparatus as defined in claim 1 or 2 in which said laser light has a wave length of 0.7 to 1.7

4. A laser light irradiating apparatus as defined in any one of claims 1 through 5 in which said laser light is Nd:YAG laser light.

5. A laser light irradiating apparatus as defined in any one of claims 1 through 4 in which said laser light is pulsed laser light having an energy of 200 mJ or more per one pulse.

6. A laser light irradiating apparatus as defined in any one of claims 1 through 5 in which said handpiece has at its front end a guide tube, said optical fiber passes through the guide tube so that it projects beyond the front end of the guide tube, said laser light absorbing liquid being caused to flow through a space between said guide tube and said optical fiber so that it is injected from an opening at the front end of the guide tube.
